(19) United States
(12) Patent Application Publication
(10) Pub. No.: US 2020/0206319 A1
(43) Pub. Date: Jul. 2, 2020

(54) METHOD FOR PREVENTING OR TREATING SEXUALLY TRANSMITTED INFECTIONS

(71) Applicants: University of Cape Town, Cape Town (ZA); University of Southampton, Southampton (GB)

(72) Inventors: Georgia Schäfer, Cape Town (ZA); William Gordon Charles Horsnell, Cape Town (ZA); Howard William Clark, Southampton (GB)

(21) Appl. No.: 16/631,281

(22) PCT Filed: Jul. 17, 2018
(86) PCT No.: PCT/IB2018/055290

§ 371 Date: Jan. 15, 2020

(30) Foreign Application Priority Data
Jul. 17, 2017 (GB) 1711423.2

(51) Int. Cl. A61K 38/17; A61P 31/12

(52) U.S. Cl. CPC A61K 38/395; A61P 31/12

(57) ABSTRACT

Surfactant protein A (SP-A) is described for preventing and/or treating a sexually transmitted infection (STI) in a subject. The STI is caused by a DNA virus, such as Human papillomavirus (HPV) and/or Herpes simplex virus (HSV). SP-A can thus be used to prevent cervical cancer, genital warts and/or genital ulcers. Pharmaceutical compositions and kits comprising SP-A are also described, as is a method for preventing and/or treating STIs caused by DNA viruses, the method comprising administering an effective amount of SP-A to a subject in need thereof.

### CROSS-REFERENCE TO RELATED APPLICATIONS

This application claims priority to United Kingdom patent application no. 1711423.2 filed on 17 Jul. 2017 which is incorporated by reference herein.

### FIELD OF THE INVENTION

The invention relates to the use of surfactant protein A (SP-A) for preventing or treating sexually transmitted infections (STIs).

### BACKGROUND TO THE INVENTION

It is estimated that over a billion people have sexually transmitted infections (STIs) other than HIV/AIDS, and that these STIs result in over 100,000 deaths each year.

Human papillomavirus (HPV) is the most common sexually transmitted infection and a principle cause of various anogenital cancers, including cervical cancer. The majority of cervical cancer cases occur in low and middle-income countries (LMIC). Prophylactic vaccines exist to combat HPV infection but accessibility to these in LMIC is limited. Alternative preventative measures against HPV infection are therefore also needed to control cervical cancer risk.

Herpes simplex virus is also a highly prevalent sexually transmitted virus which causes significant disease burden worldwide (HSV-2 is the principal cause of genital ulcers). Like HPV, HSV infection is currently incurable.

STIs can also be caused by other viruses, bacteria and parasites.

There is therefore still a need for a new treatment which prevents or reduces the risk of a subject acquiring a STI.

### SUMMARY OF THE INVENTION

According to a first embodiment, the invention provides surfactant protein A (SP-A), or a fragment, homologue, variant or derivative thereof, for use in preventing and/or treating a sexually transmitted infection (STI) in a subject, wherein the STI is caused by a DNA virus.

More particularly, the DNA virus may be human papillomavirus (HPV) and/or herpes simplex virus (HSV). The HPV may be any type of HPV, such as types 6, 11, 16, 18, 26, 31, 33, 35, 39, 45, 51, 52, 53, 56, 58, 59, 66, 68, 73 and 82. The HSV may be HSV-1 or HSV-2.

The subject may be a mammal, such as a human. More particularly, the subject may be a female human.

The SP-A may comprise the sequence shown in SEQ ID NO: 1, or the SP-A fragment, homologue, variant or derivative may comprise an amino acid sequence having at least 70% sequence identity over at least 50 amino acid residues of SEQ ID NO:1.

SP-A, or the fragment, homologue, variant or derivative thereof, may bind to HPV or HSV.

SP-A, or the fragment, homologue, variant or derivative thereof may neutralize the ability of HPV or HSV to successfully infect the host.

SP-A bound HPV or SP-A bound HSV may be preferentially internalised by immune cells (such as macrophages).

The SP-A, or the fragment, homologue, variant or derivative thereof, may be administered to the genital area or reproductive tract of the subject.

The SP-A, or the fragment, homologue, variant or derivative thereof, may be administered in combination with an antimicrobial therapy.

In a second embodiment, the invention provides a nucleic acid encoding SP-A, or a fragment, homologue, variant or derivative thereof, for use in preventing and/or treating a STI in a subject, wherein the STI is caused by a DNA virus.

In a third embodiment, the invention provides a pharmaceutical composition comprising SP-A, or a fragment, homologue, variant or derivative thereof, for use in preventing and/or treating a STI in a subject, wherein the STI is caused by a DNA virus.

The composition may further comprise a pharmaceutical excipient and/or carrier.

In a further embodiment, the invention provides the use of SP-A, or a fragment, homologue, variant or derivative thereof, in the manufacture of a medicament for preventing and/or treating a STI in a subject, wherein the STI is caused by a DNA virus.

In a further embodiment, the invention provides a kit comprising SP-A, or a fragment, homologue, variant or derivative thereof, for use in preventing and/or treating a STI, wherein the STI is caused by a DNA virus.

The kit may optionally be in the form of a pharmaceutical combination further comprising an antimicrobial therapy and/or pharmaceutical composition.

The kit may include an applicator for administering the SP-A into the reproductive tract.

In yet a further embodiment, the invention provides a method for preventing and/or treating a STI in a subject, wherein the STI is a DNA virus, the method comprising a step of administering SP-A, or a fragment, homologue, variant or derivative thereof, to the subject.

Further embodiments of the invention extend to SP-A, a nucleic acid encoding SP-A, or a pharmaceutic composition or kit comprising SP-A, for use in preventing cervical cancer, genital warts and/or genital ulcers; to the use of SP-A in the manufacture of a medicament for preventing cervical cancer, genital warts and/or genital ulcers; and to a method for preventing cervical cancer, genital warts and genital ulcers in a subject, the method comprising administering SP-A to the subject.

### BRIEF DESCRIPTION OF THE FIGURES

FIG. 1 shows that binding of HPV16-PsVs to SP-A but not SP-D results in increased viral uptake by RAW264.7 macrophages but not HeLa cervical epithelial cells. A-C) Co-immunoprecipitation experiments displaying the input, flow through (FT) and eluate samples of (A) HPV16-PsVs and SP-A alone (controls), (B) HPV16-PsVs and SP-A together and (C) HPV16-PsVs and SP-D together.

FIG. 2 shows that uptake of SP-A-mediated HPV16-PsVs by RAW264.7 macrophages is calcium-dependent, but not dependent on the CRD.

FIG. 3 shows that infection of C57BL/6 mice with HPV16-PsVs does not alter SP-A expression.

FIG. 4 shows that SP-A reduces HPV16-PsVs infection in C57BL/6 mice.

### DETAILED DESCRIPTION OF THE INVENTION

Surfactant protein A (SP-A) has been identified and characterised previously, in for example, Wright, J. R. et al., “Surfactant apoprotein Mr=26,000-36,000 enhances uptake of liposomes by type II cells”, J Biol Chem 1987 262(6):2888-94. SP-A is an innate immune system collectin which is primarily expressed in the lungs and facilitates phagocytosis of e.g. microbes by alveolar macrophages through opsonisation. The function of SP-A proteins is primarily understood in the lung, but they are also expressed at other sites of the body, including the female reproductive tract.

The inventors have now surprisingly found that administration of SP-A to the reproductive tract of a subject reduces the risk of the subject acquiring a sexually transmitted infection (STI) caused by a DNA virus such as HPV or HSV.

Innate mucosal immune responses in the female reproductive tract, the first barrier associated with clearance of incoming HPV, are an underexplored area. Due to several immune evasion mechanisms, infiltration and activation of macrophages and dendritic cells (as the most likely antigen presenting cells) upon HPV infection are usually relatively ineffective, thereby leading to inept humoral and cellular immunity.

The inventors have identified that host innate immunity which protects against Human papillomavirus (HPV) can be enhanced by raising the levels of SP-A in the female reproductive tract. Enhanced immune recognition of HPV reduces the risk of the virus reaching squamous epithelial cells where it could establish a persistent infection, thereby increasing the risk of viral induction of invasive cancer. Raising levels of SP-A in the female reproductive tract could therefore lead to reduced HPV infection and other STI infections and their pathological consequences.

More specifically, the examples below show that:
SP-A binds to HPV;
SP-A bound HPV is preferentially internalised by macrophages; and
HPV infection in a mouse model is reduced when exogenous SP-A is added to the female reproductive tract.

SP-A may also bind to HSV (e.g. HSV-1 or HSV-2) and lead to reduced HSV-infection in a subject.

SP-A was therefore identified as being suitable for use in topical microbicides which provide protection against viral infections of the female reproductive tract, and in particular against DNA viral infections.

As used herein, SP-A refers to any SP-A polypeptide or nucleic acid (as the context requires). The SP-A polypeptide or nucleic acid for use according to the present invention may be a human SP-A having the NCBI Reference Sequence: NG_021189 or the GenBank accession number NM_005411.

The amino acid sequence of such a human SP-A is shown in SEQ ID NO: 1, and two examples of nucleic acid sequences encoding human SP-A are shown in SEQ ID NOs: 2 and 3.

SEQ ID NO: 1
MWLCPLALNL ILMAASGAVC EVKDVCVGSP GIPGTPGSHG LPGRDGRDGL KGDPGPPGPM
GPPGEMPCPP GNDGLPGAPG IPGECGEKGE PGERGPPGLP AHLDEELQAT LHDFRHQILQ
TRGALSLQGS IMTVGEKVFS SNGQSITFDA IQEACARAGG RIAVPRNPEE NEAIASFVKK
YNTYAYVGLT EGPSPGDFRY SDGTPVNYTN WYRGEPAGRG KEQCVEMYTD GQWNDRNCLY
SRLTICEF

The amino acid sequence of SP-A may be lacking the signal sequence (e.g. the amino acid sequence may lack residues 1 to 20 of SEQ ID NO: 1).

| SEQ ID NO: 2 | |
|---|---|
| 1 | gacttggagg cagagaccca agcagctgga ggctctgtgt gtgggtgagt ttagccccat |
| 61 | cccctaggtg ttctccagct tgaggatcgc aggcagagag gaccagccca gcagccacag |
| 121 | gcctgaccaa agcccaggct gggaaggagg gcaactcccc attttccact gggaggtgtt |
| 181 | tcacagcaca gtcaacatag gtgacctgca aagatcctca tgtttgttat tttctttggc |
| 241 | cagatccatc cctacagggt tcagcagggc ctacaggagg ggcagtgaga gaacagaccc |
| 301 | caaaaagaaa ggggactcca tgactgacca ccttgagggg ggccaggctg cgggccccgt |
| 361 | tcatcttttt tcattctcag gtcgctgatt tcttggagcc tgaaaagaaa gtaacacagc |
| 421 | agggatgagg acagatggtg tgagtcagtg agtgagtgac ctgactaata gcctgggagg |
| 481 | gacagggcag gttttctgca gagcacggaa gattcagctg aagtcagaga ggtgaagcca |
| 541 | gtttcccagg gtaacatagt gaggcactga aagaaaggag actgcactgg agcccaggtc |
| 601 | cccgggctcc ccagagctcc ttactcttcc tcctcctcag cagcctggag accccacaac |
| 661 | ctccagccgg aggcctgaag catgaggcca tgccaggtgc caggtgatgc tgggaatttt |
| 721 | cccgggagct tcgggtcttc ccagcactct ggtctcgccc gccctgcctc tcgggctctg |
| 781 | cccagcttcc tgagtcctga cagagcacag tgggggagat gttggcagag gtggcagatg |
| 841 | ggctcacggc catccctcct gcaggagcag cgactggacc cagagccatg tggctgtgcc |
| 901 | ctctggccct caacctcatc ttgatggcag cctctggtgc tgtgtgcgaa gtgaaggacg |
| 961 | tttgtgttgg aagccctggt atccccggca ctcctggatc ccacggcctg ccaggcaggg |
| 1021 | acgggagaga tggtctcaaa ggagaccctg gccctccagg tactgtgctg cagaccccac |
| 1081 | cctcagctga gggacacaga ccccttttca ggaggcccat ctgtccaggc ccctaggctg |
| 1141 | tgggccatag tgagctgggg gctatagtaa gctgggtggg acttcagtct gcagggctgg |
| 1201 | tgggttcctg gggcccttat gatggcgcat cctggagagt ctgtcctcat agtgcccacg |
| 1261 | gagtgataga gtgatagctg agccagccct ggtgataatg ggcatcgagt ctcactagct |
| 1321 | ccaaccagtt gtgggtgaca gatcctacac atccatgtct cttttctctg caggccccat |
| 1381 | gggtccacct ggagaaatgc catgtcctcc tggaaatgat gggctgcctg gagcccctgg |
| 1441 | tatccctgga gagtgtggag agaaggggga gcctggcgag aggggccctc caggtgagca |
| 1501 | gggtggggca ggtgggcagt ggaaacatgg gcacagcgac cctgaagtca gttacacggg |
| 1561 | gatgatgggg atcagacaaa ccctacaggt tccccaaggg catttggctc aacctaagta |
| 1621 | agagaggata agcttgaggg agaaagctga ggtgtctggg gagtgtggtc acaattcagg |
| 1681 | gaaaggcagg tgtgggaagt cctccgtgcc tcatgaccac cgatggggac acactgagtc |
| 1741 | aggtgtggga tgagggacag cactgggagg caggggaggc atgtcctggg atggaggccc |
| 1801 | tgggggctgt ctgaagggtg aatgcggacg aggcatccag acagacggtg tgatcaggag |
| 1861 | ccccacagac agaggggaac tttgaagctc agagcggtaa gcaagtccat cagggcagtg |
| 1921 | cagagagcat catgcttgcc cttggtggag ggtgcgggag agggacttgc cccacagagg |
| 1981 | cgggcagaca gaacccctcg agggacagag caggaaagag gacaaggggt gggggtctca |
| 2041 | gcaggggcaa ggcttcacta aagaataggg gaccacgggg tgtggagaca cactggaatc |
| 2101 | ttgtggaccc tctgagccta gggtctgggt ggcgcctaac agcaatgaaa gggcagagtt |
| 2161 | ccaggattgc agatggcaaa acacctgcgt ggcagcaagt gggagtcttc actggcctgc |
| 2221 | ccctccttct gtgtggggca ctctccacag ggcttccagc tcatctagat gaggagctcc |
| 2281 | aagccacact ccacgacttt agacatcaaa tcctgcagac aaggggaggt aaggggaccc |
| 2341 | cctgggcctc acggggtagg agtttcccac aaattcccct cattctcagc accagcttct |
| 2401 | agaacataga gattacaaat aggcatgcac atgcaggtct tggggaaagg aatgacgctt |
| 2461 | gcttttctga tgtctttgaa tggcccagag gagacagaag cagacacaat tcactcccca |
| 2521 | tttcatagga aagcaagttc tccacctgcc ttgctttcca ctgaattcca ggaaattgca |
| 2581 | ccatttctgg caataagtaa ttgttactta ggtgaatgaa taaatggagg agagtctaaa |
| 2641 | agtgaattta gaaaactgca attggaagag gaagagaaga cacagagaga ggcagagatg |
| 2701 | gagagactgg ggagaatctg gtagcagaga ccccaggtga gggaggtggc ttagagacaa |
| 2761 | agtggtcagt ggcctgaccc ggactcctct gctctcagcc ctcagtctgc agggctccat |
| 2821 | aatgacagta ggagagaagg tcttctccag caatgggcag tccatcactt ttgatgccat |
| 2881 | tcaggaggca tgtgccagag caggcggccg cattgctgtc ccaaggaatc cagaggaaaa |
| 2941 | tgaggccatt gcaagcttcg tgaagaagta caacacatat gcctatgtag gcctgactga |
| 3001 | gggtcccagc cctggagact tccgctactc agacgggacc cctgtaaact acaccaactg |
| 3061 | gtaccgaggg gagcccgcag gtcggggaaa agagcagtgt gtggagatgt acacagatgg |
| 3121 | gcagtggaat gacaggaact gcctgtactc ccgactgacc atctgtgagt tctgagaggc |
| 3181 | atttaggcca tgggacaggg aggacgctct ctggccttcg gcctccatcc tgaggctcca |
| 3241 | cttggtctgt gagatgctag aactcccttt caacagaatt cacttgtggc tattgggact |
| 3301 | ggaggcaccc ttagccactt cattcctctg atgggccctg actcttcccc ataatcactg |
| 3361 | accagccttg acactcccct tgcaaactct cccagcactg caccccaggc agccactctt |
| 3421 | agccttggcc ttcgacatga gatggagccc tccttattcc ccatctggtc cagttccttc |
| 3481 | acttacagat ggcagcagtg aggtcttggg gtagaaggac cctccaaagt cacacaaagt |
| 3541 | gcctgcctcc tggtcccctc agctctctct ctgcaaccca gtgccatcag gatgagcaat |
| 3601 | cctggccaag cataatgaca gagagaggca gacttcgggg aagccctgac tgtgcagagc |
| 3661 | taaggacaca gtggagattc tctggcactc tgaggtctct gtggcaggcc tggtcaggct |
| 3721 | ctccatgagg ttagaaggcc aggtagtgtt ccagcagggt ggtggccaag ccaaccccat |
| 3781 | gattgatgtg tacgattcac tcctttgagt ctttgaatgg caactcagcc ccctgacctg |
| 3841 | aagacagcca gcctaggcct ctagggtgac ctagagccgc cttcagatgt gacccgagta |
| 3901 | actttcaact gatgaacaaa tctgcaccct acttcagatt tcagtgggca ttcacaccac |
| 3961 | cccccacacc actggctctg ctttctcctt tcattaatcc attcacccag atatttcatt |
| 4021 | aaaattatca cgtgccaggt cttaggatat gtcgtggggt gggcaaggta atcagtgaca |
| 4081 | gttgaagatt tttttttccc agagcttatg tcttcatctg tgaaatggga ataagatact |
| 4141 | tgttgctgtc acagttatta ccatcccccc agctaccaaa attactacca gaactgttac |
| 4201 | tatacacaga ggctattgac tgagcaccta tcatttgcca agaaccttga caagcacttc |
| 4261 | taatacagca tattatgtac tattcaatct ttacacaatg tcacgggacc agtattgttt |
| 4321 | cctcattttt tataaggaca ctgaagcttg gaggagttaa atgttttgag tattattcca |
| 4381 | gagagcaagt ggcagaggct ggatccaaac ccatcttcct ggacctgaag cttatgcttc |
| 4441 | cagccacccc actcctgagc tgaataaaga tgatttaagc ttaataaatc gtgaatgtgt |
| 4501 | tcaca |

SEQ ID NO: 3
gacttggagg cagagaccca agcagctgga ggctctgtgt gtgggtcgct gatttcttgg
agcctgaaaa gaaagtaaca cagcagggat gaggacagat ggtgtgagtc agtgagagca
gcgactggac ccagagccat gtggctgtgc cctctggccc tcaacctcat cttgatggca
gcctctggtg ctgtgtgcga agtgaaggac gtttgtgttg gaagccctgg tatccccggc
actcctggat cccacggcct gccaggcagg gacgggagag atggtctcaa aggagaccct
ggccctccag gccccatggg tccacctgga gaaatgccat gtcctcctgg aaatgatggg
ctgcctggag cccctggtat ccctggagag tgtggagaga agggggagcc tggcgagagg
ggccctccag ggcttccagc tcatctagat gaggagctcc aagccacact ccacgacttt
agacatcaaa tcctgcagac aaggggagcc ctcagtctgc agggctccat aatgacagta
ggagagaagg tcttctccag caatgggcag tccatcactt ttgatgccat tcaggaggca
tgtgccagag caggcggccg cattgctgtc ccaaggaatc cagaggaaaa tgaggccatt
gcaagcttcg tgaagaagta caacacatat gcctatgtag gcctgactga gggtcccagc
cctggagact tccgctactc agacgggacc cctgtaaact acaccaactg gtaccgaggg
gagcccgcag gtcggggaaa agagcagtgt gtggagatgt acacagatgg gcagtggaat
gacaggaact gcctgtactc ccgactgacc atctgtgagt tctgagaggc atttaggcca
tgggacaggg aggacgctct ctggccttcg gcctccatcc tgaggctcca cttggtctgt
gagatgctag aactcccttt caacagaatt cacttgtggc tattgggact ggaggcaccc
ttagccactt cattcctctg atgggccctg actcttcccc ataatcactg accagccttg
acactcccct tgcaaactct cccagcactg caccccaggc agccactctt agccttggcc
ttcgacatga gatggagccc tccttattcc ccatctggtc cagttccttc acttacagat
ggcagcagtg aggtcttggg gtagaaggac cctccaaagt cacacaaagt gcctgcctcc
tggtcccctc agctctctct ctgcaaccca gtgccatcag gatgagcaat cctggccaag
cataatgaca gagagaggca gacttcgggg aagccctgac tgtgcagagc taaggacaca
gtggagattc tctggcactc tgaggtctct gtggcaggcc tggtcaggct ctccatgagg
ttagaaggcc aggtagtgtt ccagcagggt ggtggccaag ccaaccccat gattgatgtg
tacgattcac tcctttgagt ctttgaatgg caactcagcc ccctgacctg aagacagcca
gcctaggcct ctagggtgac ctagagccgc cttcagatgt gacccgagta actttcaact
gatgaacaaa tctgcaccct acttcagatt tcagtgggca ttcacaccac cccccacacc
actggctctg ctttctcctt tcattaatcc attcacccag atatttcatt aaaattatca
cgtgccaggt cttaggatat gtcgtggggt gggcaaggta atcagtgaca gttgaagatt
tttttttccc agagcttatg tcttcatctg tgaaatggga ataagatact tgttgctgtc
acagttatta ccatcccccc agctaccaaa attactacca gaactgttac tatacacaga
ggctattgac tgagcaccta tcatttgcca agaaccttga caagcacttc taatacagca
tattatgtac tattcaatct ttacacaatg tcacgggacc agtattgttt cctcattttt
tataaggaca ctgaagcttg gaggagttaa atgttttgag tattattcca gagagcaagt
ggcagaggct ggatccaaac ccatcttcct ggacctgaag cttatgcttc cagccacccc
actcctgagc tgaataaaga tgatttaagc ttaataaatc gtgaatgtgt tcacaaaaaa
aaaaaaaaaa

The present invention provides a SP-A polypeptide, variant, homologue, fragment or derivative for use in preventing and/or treating one or more STIs in a subject.

The terms “variant” or “derivative” in relation to the amino acid sequences for use according to the present invention includes any substitution of, variation of, modification of, replacement of, deletion of or addition of one (or more) amino acids from or to the sequence providing the resultant amino acid sequence retains substantially the same activity as the unmodified sequence, preferably having at least the same activity as the SP-A polypeptide shown in SEQ ID NO: 1.

Polypeptides having the amino acid sequence of SEQ ID NO: 1, or fragments or homologues thereof may be modified for use as described herein. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides described here.

Conservative substitutions may be made, for example according to Table 1. Amino acids in the same block in the second column and preferably in the same line in the third column may be substituted for each other:

TABLE 1
Conservative amino acid substitutions

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Preferred fragments include those having one or more biological activities of SP-A.

SP-A polypeptides also generally include any recombinant fragment of SP-A.

The SP-A, SP-A polypeptide or SP-A fragment for use according to the present invention also includes homologous sequences obtained from any source, for example related viral/bacterial proteins, cellular homologues and synthetic peptides, as well as variants or derivatives thereof.

Thus polypeptides also include those encoding homologues of SP-A from other species including animals such as mammals (e.g. mice, rats or rabbits), especially primates, more especially humans. More specifically, homologues include human homologues.

Thus, the SP-A for use according to the present invention may be a variant, homologue or derivative of the amino acid sequence of the SP-A sequence shown in SEQ ID NO: 1, as well as a variant, homologue or derivative of a nucleotide sequence encoding the amino acid sequence.

As used herein, a homologous sequence is taken to include an amino acid sequence which is at least 15, 20, 25, 30, 40, 50, 60, 70, 80 or 90% identical, preferably at least 95 or 98% identical at the amino acid level over at least 50 or 100, preferably 200 amino acids with the sequence of SP-A shown in SEQ ID NO: 1. In particular, homology should typically be considered with respect to those regions of the sequence known to be essential for protein function rather than non-essential neighbouring sequences. This is especially important when considering homologous sequences from distantly related organisms.

Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These publicly and commercially available computer programs can calculate % homology between two or more sequences. % homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestf it package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The variants, homologues, fragments or derivatives of SP-A for use according to the present invention may encompass related polypeptides which provide one or more of the biological activities of SP-A.

The SP-A polypeptides, variants, homologues, fragments and derivatives for use as described herein may be in a substantially isolated form. It will be understood that such polypeptides may be mixed with carriers or diluents which will not interfere with the intended purpose of the protein and still be regarded as substantially isolated. A SP-A variant, homologue, fragment or derivative may also be in a substantially purified form, in which case it will generally comprise the protein in a preparation in which more than 90%, e.g. 95%, 98% or 99% of the protein in the preparation is a protein.

As used herein, the terms “polynucleotide”, “nucleotide”, and nucleic acid are intended to be synonymous with each other. “Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

SP-A nucleic acids, variants, fragments, derivatives and homologues may comprise DNA or RNA. They may be single-stranded or double-stranded. They may also be polynucleotides which include within them synthetic or modified nucleotides. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence. Preferably said variant, homologues or derivatives code for a polypeptide having biological activity.

As indicated above, with respect to sequence homology, preferably there is at least 50 or 75%, more preferably at least 85%, more preferably at least 90% homology to SEQ ID NO: 2 or SEQ ID NO: 3. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and -9 for each mismatch. The default gap creation penalty is −50 and the default gap extension penalty is −3 for each nucleotide.

The nucleic acid sequence may have at least 80, 85, 90, 95, 98 or 99% identity to the sequence shown as SEQ ID NO. 2 or SEQ ID NO: 3, provided that it encodes a SP-A polypeptide suitable for use as defined in the first embodiment of the invention.

SP-A can be used in order to prevent or treat sexually transmitted infections caused by DNA viruses. Examples of DNA viruses causing STIs are Human papillomavirus (HPV), Herpes simplex virus (HSV) and Molluscum contagiosum virus (MCV). These viruses are classified in virus Group 1 (dsDNA) and are distinguishable from HIV (Group VI—ssRNA-RT viruses) and Hepatitis B virus (HBV) and Hepatitis C virus (HCV) (Group VII—dsDNA-RT viruses). Viruses such as HIV are therefore excluded from the present invention. The SP-A may neutralize the ability of the virus to successfully infect the host.

Treatment with SP-A is effective against any type of HPV.

The SP-A can be used as a broad spectrum treatment for preventing or treating both HPV and HSV infection. Moreover, as the binding of the SP-A to HPV is not dependent on the type of HPV, it can be used to prevent or treat a broad range of HPV types, including HPV types 6 and 11 (which cause genital warts and laryngeal papillomatosis) and HPV types 16, 18, 26, 31, 33, 35, 39, 45, 51, 52, 53, 56, 58, 59, 66, 68, 73 and 82 (which are carcinogenic). Thus, SP-A can be used to prevent cervical and other cancers (including cancer of the vulva and vagina), genital warts and genital ulcers (caused by HSV).

When used for the prevention of STIs, the invention relates to the prophylactic use of SP-A. In this aspect, exogenous SP-A may be administered to a subject who has not yet contracted the infection and/or who is not showing any symptoms of disease associated with the infection to prevent or impair the cause of the infection or to reduce or prevent development of at least one symptom associated with the infection.

When used for the treatment of STIs, the invention relates to the therapeutic use of SP-A. Herein exogenous SP-A may be administered to a subject having an existing infection in order to lessen, reduce or improve at least one symptom associated with the infection and/or to slow down, reduce or block the progression of the infection.

The SP-A can be administered on its own or in combination with an additional treatment. For example, the additional treatment could be an antimicrobial formulation containing a microbicide such as an antiretroviral (e.g. tenofovir, dapivirine or UC-781), cellulose sulphate, dextrin sulphate, nonoxynol-9, carrageenan or Lactobacillus crispatus. Addition of SP-A to the formulation could enhance its antimicrobial activity.

The administration of SP-A can be accomplished using any of a variety of routes that make the active ingredient bioavailable. For example, the SP-A can be formulated into a topical composition in the form of a gel, cream, lotion, aerosol spray, film, suppository or vaginal ring for insertion into the vagina or rectum, or could be in a formulation for oral administration, such as a tablet or syrup.

Preferably, SP-A is administered such that it is available in an active form in the reproductive tract of the subject to which it is administered.

Typically, a physician will determine the actual dosage that is most suitable for an individual subject and it will vary with the age, weight and response of the particular patient. The dosage is such that it is sufficient to prevent and/or treat an STI.

The present invention also provides a pharmaceutical composition comprising SP-A for use in preventing and/or treating STIs caused by a DNA virus.

SP-A may be administered with a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents.

The present invention also provides a kit comprising SP-A for use in preventing and/or treating one or more STIs caused by DNA viruses. The kit comprises SP-A as defined above, and may be in the form of a pharmaceutical combination further comprising an antimicrobial treatment and/or pharmaceutical composition as defined above. The kit may also include an applicator for administering the SP-A into the reproductive tract.

The present invention further relates to a method for preventing and/or treating one or more STIs caused by a DNA virus, the method comprising the step of administering exogenous SP-A to a subject. The method may also comprise the use of an antimicrobial therapy and/or a pharmaceutical composition as defined above.

The present invention also relates to use of SP-A in the manufacture of a medicament for preventing and/or treating a STI in a subject, wherein the STI is caused by a DNA virus.

The invention will now be described in more detail by way of the following non-limiting examples.

### Examples

### Binding of SP-A to HPV

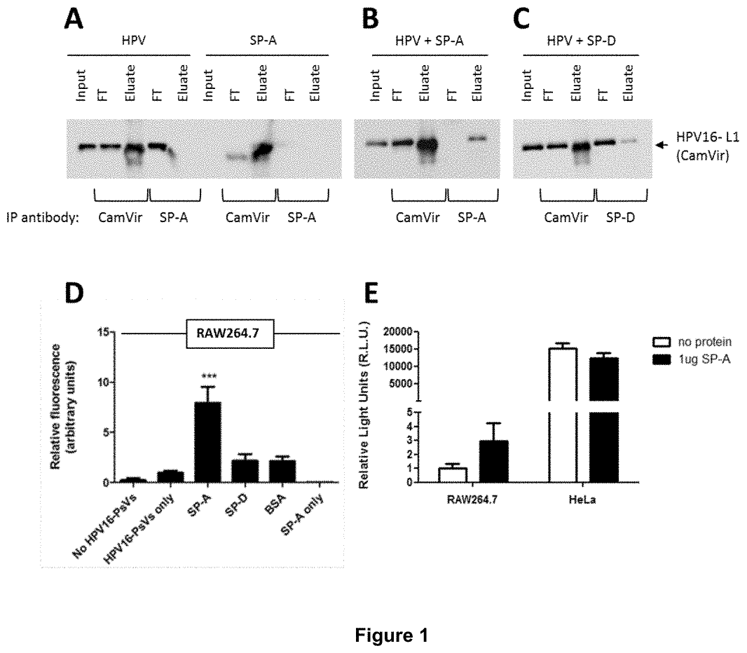

Co-immunoprecipitation and flow cytometry experiments were conducted to determine whether SP-A and the closely related surfactant protein D (SP-D) bind to HPV16 pseudovirions (HPV16-PsVs) (1-3). Native human SP-A purified from bronchoalveolar lavage fluid (4) was used. Co-immunoprecipitation experiments displaying the input, flow through (FT) and eluate samples of (A) HPV16-PsVs and SP-A alone (controls) (FIG. 1A), HPV16-PsVs and SP-A together (FIG. 1B) and HPV16-PsVs and SP-D together (FIG. 10). Complexes were incubated for 1 h at 4° C., followed by precipitations with either anti-HPV16-L1 (CamVir), anti-SP-A or anti-SP-D antibodies. Western Blots were performed using the CamVir antibody to detect the presence of HPV16-L1.

SP-A bound HPV16-PsVs and the resulting HPV16-SP-A complex showed enhanced uptake by RAW264.7 murine macrophages. SP-D bound HPV16-PsVs weakly and had no effect on viral uptake.

SP-A was found to enhance viral recognition of HPV, and both impairs initial infection and stimulates anti HPV host innate immunity.

### Functional Virus Assays

RAW264.7 cells were infected with fluorescently labelled HPV16-PsVs (pre-absorbed with purified proteins where indicated) for 1h at 37° C. (FIG. 1 D). Cells were washed extensively and lifted with trypsin/EDTA to remove surface-bound virions, thereby allowing detection of internalised viral particles by flow cytometry. Experiments were performed in triplicates, quantified by quadrant analysis of the dot plot of three independent experiments and presented as x-fold increase relative to the mean fluorescence intensity of cells infected with untreated HPV16-PsVs which was set as 1. Significances were calculated by means of one-way ANOVA and Tukey post-hoc tests. ***indicates statistical significance between uptake of HPV16-PsVs in the presence of SP-A as compared to the other tested conditions (***=p<0.001). The murine macrophage cell line RAW264.7 and the human epithelial cell line HeLa were infected with HPV16-PsVs encapsidating the firefly luciferase reporter plasmid pGL3 for 48h (FIG. 1E). Where indicated, the viral particles were pre-absorbed with purified SP-A protein before infection. Firefly luciferase activities in the cell lysates as a measure for successful infection is presented as Relative Light Units relative to RAW264.7 cells infected with untreated HPV16-PsVs which was set as 1.

Infection of RAW264.7 macrophages with fluorescently labelled HPV16-PsVs for 1h at 37° C. demonstrated that pre-incubation of viral particles with recombinant SP-A but not SP-D enhanced uptake by RAW264.7 cells (FIG. 1(D)). Enhanced infectious internalisation of SP-A-coated HPV16-PsVs was only seen in RAW264.7 macrophages but not HeLa epithelial cells (FIG. 1(E)).

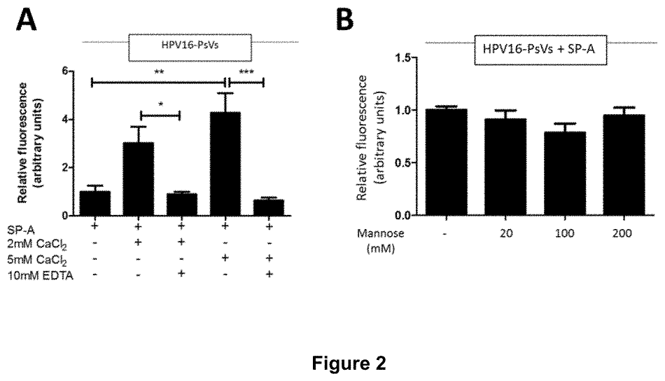

AF488-HPV16-PsVs were incubated in the presence of 10-fold (w/w) excess SP-A and with or without 2 mM or 5 mM calcium chloride in the presence or absence of 10 mM EDTA as indicated (FIG. 2A), or with various concentrations of mannose (FIG. 2B). Shown are summaries of viral internalisation experiments as determined by FACS as described in FIG. 1D. Fluorescent readings are displayed relative to HPV16-PsVs internalisation in the presence of SP-A only which was set as 1. Quantification of three independent experiments performed in duplicate are shown. Statistical significance was determined using one-way ANOVA and Tukey post-hoc tests. *=p<0.05, **=p<0.01, ***=p<0.001. Bars depict mean values, with error bars showing SEM.

### Effect of Exogenous SP-A on HPV16-PsVs Infection Levels in Mice

To assess if this relationship persisted in vivo HPV16-PsVs infections were carried out using the well-established murine HPV16-PsVs cervicovaginal challenge model system (Roberts et al., Longet et al.). Surprisingly, neither naïve nor C57BL/6 mice challenged with HPV16-PsVs expressed SP-A in the female genital tract. However, pre-incubation of HPV16-PsVs with purified SP-A at a 1:10 weight per weight ratio reducted the level of HPV16-PsV infection.

Female wildtype C57/BL6 mice were assessed for SP-A expression in various organs and body fluids. FIG. 2(A) shows a Western Blot probed for SP-A in the lung, bronchoalveolar lavage fluid (BAL), genital tract tissue (GT), and vaginal lavage fluid. 20 μg of each sample was loaded per lane, while 0.5 μg of purified human SP-A protein was loaded as control. No endogenous SP-A was detected in the female genital tract.

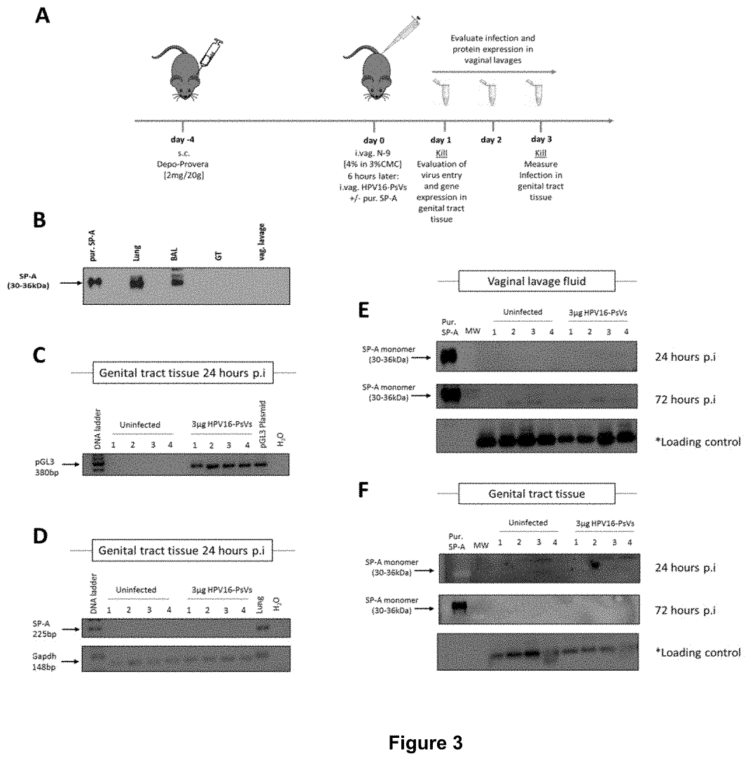

FIG. 3A shows a mouse model for HPV16-PsVs infection using C57BL/6 mice, adapted from Roberts et al., 2007 (Nature Med). Briefly, 6-10 weeks old female wildtype C57/BL6 mice per group were injected with 2 mg Depo-Provera (s.c.) for 4 days, and then pre-treated with 25 μl 4% N9 in 3% CMC i.vag. for 6h prior to HPV16-PsVs infection. 4-6 mice per group were i.vag. infected with the viral particles encapsidating the reporter gene firefly luciferase (1 μg=3×10¹⁰ IU). After 1-3 days, tissues were harvested for analysis. Western Blot assessing SP-A expression in various organs and body fluids of naïve female wildtype C57BL/6 mice is shown in FIG. 3B. No endogenous SP-A was detected in the female genital tract of naïve mice. Four mice per group were i.vag. infected with 3 μg HPV16-PsVs encapsidating the pGL3 reporter plasmid. Genital tract tissue was harvested 24 hours later and RNA extracted for gene expression analysis to confirm successful HPV infection (FIG. 3C). RNA from C) was assessed for SP-A expression, with lung RNA used as positive control. Gapdh was used as a reference gene (FIG. 3D). Four mice per group were i.vag. infected with 3 μg HPV16-PsVs. Vaginal lavages were performed 24 and 72 hours p.i. and assessed by Western Blot for the presence of SP-A. 20 μg of each sample was loaded per lane, while 0.5 μg purified human SP-A protein was loaded as control. *Loading control corresponds to a 45 kDa cellular protein that cross-reacts with the CamVir primary antibody (Shafti-Keramat et al., 2003, J Virol) (FIG. 3E).

Four mice per group were i.vag. infected with 3 μg HPV16-PsVs and genital tract tissue was harvested 24 hours or 72 hours p.i., homogenised and assessed by Western Blot for the presence of SP-A as described in E) (FIG. 3E).

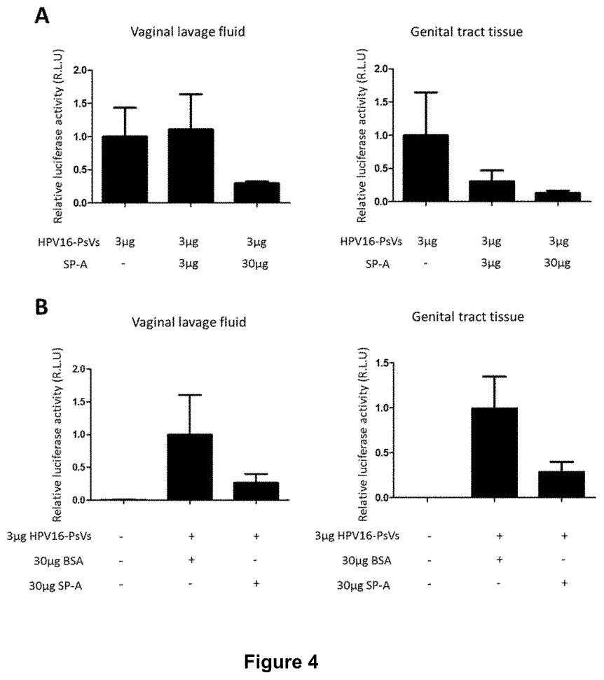

6-10 weeks old female wildtype C57BL/6 mice per group were pre-treated as described in FIG. 3A. HPV16-PsVs encapsidating firefly luciferase were pre-incubated with increasing amounts of purified SP-A protein for 1 hour on ice (FIG. 4A). 3 μg HPV16-PsVs were pre-incubated with 30 μg purified SP-A protein or 30 μg BSA for 1 hour on ice (FIG. 4B).

Mice were euthanised 72 hours p.i., and tissue harvested for analysis. Firefly luciferase activity was measured in vaginal lavage fluid (left panels) and homogenised genital tract tissue (right panels). Data were normalised to total protein in the samples and are presented as x-fold to average control (no SP-A) which was set as 1. FIG. 4A shows the data from one experiment with four mice per condition. FIG. 4B shows pooled data from two independent experiments, with a total of ten mice per condition. Bars depict mean values, with error bars showing SEM.

Addition of exogenous SP-A into the female reproductive tract was thus shown to reduce the level of HPV16-PsVs infection.

It appears that SP-A binds to HPV in a type-unspecific manner, leading to enhanced uptake by macrophages, thereby providing protection against a wide range of incoming HPV particles.

Thus, while current prophylactic vaccines type-specifically prevent new HPV infections, SP-A as an innate immune modulator may broadly interact with incoming pathogens. SP-A therefore represents a tractable antiviral candidate for preventing HPV and other related viral infections and/or viral shedding in the genital tract.

### Binding of SP-A to HSV

Studies will be conducted on the binding of SP-A to HSV. These will be carried out as described above but using live HSV instead of HPV-PsVs.

Based on previous studies by the applicant (results now shown), it is expected that SP-A will also be shown to bind to HSV and thereby reduce the risk of a subject who is exposed to HSV from becoming infected with the virus. However, final data confirming this expectation is not yet available.

### References

1. Buck C B, Pastrana D V, Lowy D R, Schiller J T. 2005. Generation of HPV pseudovirions using transfection and their use in neutralization assays. Methods in molecular medicine 119:445-462.
2. Schäfer G, Kabanda S, van Rooyen B, Marusic M B, Banks L, Parker M I. 2013. The role of inflammation in HPV infection of the Oesophagus. BMC Cancer 13:185.
3. Schäfer G, Graham L M, Lang D, Blumenthal M J, Bergant Marusic M, Katz A A. 2017. Vimentin modulates infectious internalisation of HPV16 pseudovirions. Journal of virology.
4. Wright J R, Wager R E, Hawgood S, Dobbs L, Clements J A. 1987. Surfactant apoprotein Mr=26,000-36,000 enhances uptake of liposomes by type II cells. J Biol Chem 262(6):2888-94.
5. Longet S, Schiller J T, Bobst M, Jichlinski P, Nardelli-Haefliger D. A Murine Genital-Challenge. 2011 Model Is a Sensitive Measure of Protective Antibodies against Human Papillomavirus Infection. J Virology 85(24): 13253-13259.
6. Roberts J N, Buck C B, Thompson C D, Kines R, Bernardo M, Choyke P L, Lowy D R, Schiller J T. Genital transmission of HPV in a mouse model is potentiated by nonoxynol-9 and inhibited by carrageenan. Nat Med. 2007 13(7):857-61.

1-13. (canceled)

14. A pharmaceutical composition comprising surfactant protein A (SP-A) or a fragment, homologue, variant or derivative thereof, for use in preventing and/or treating one or more sexually transmitted infections (STIs) in a subject, wherein the STI is a DNA virus infection.

15. The pharmaceutical composition of claim 14, wherein the DNA virus is Human papillomavirus (HPV) and/or Herpes simplex virus (HSV).

16. The pharmaceutical composition of claim 14, which further comprises a pharmaceutical excipient and/or carrier.

17. The pharmaceutical composition of claim 14, which further comprises a microbicide.

18. The pharmaceutical composition of claim 14, which is formulated for delivery into the female reproductive tract.

19. The pharmaceutical composition of claim 14, which is formulated for topical application or as a suppository.

20-22. (canceled)

23. A kit comprising SP-A or a fragment, homologue, variant or derivative thereof, for use in preventing and/or treating one or more STIs, wherein the STI is a DNA virus infection.

24. The kit of claim 23, wherein the DNA virus is Human papillomavirus (HPV) and/or Herpes simplex virus (HSV).

25. The kit of claim 23, which further comprises a microbicide.

26. The kit of claim 23, which further comprises an applicator for administering the SP-A into the reproductive tract.

27. A method for preventing and/or treating at least one STI, cervical cancer, genital warts or genital ulcers in a subject, wherein the STI is a DNA virus infection, the method comprising a step of administering an effective amount of SP-A or a fragment, homologue, variant or derivative thereof to the subject.

28. The method of claim 27, wherein the DNA virus is Human papillomavirus (HPV) and/or Herpes simplex virus 2 (HSV-2).

29. The method of claim 27, wherein the SP-A or fragment, homologue, variant or derivative thereof is administered to the genital area or reproductive tract of the subject.

30. The method of claim 29, wherein the SP-A or fragment, homologue, variant or derivative thereof is administered in combination with a microbicide.

31-36. (canceled)


SEQUENCE LISTING (3 sequences)

SEQ ID NO: 1 - protein, 248 aa, Homo sapiens
MWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 2 - DNA, 4505 bp, Homo sapiens
gacttggaggcagagacccaagcagctggaggctctgtgtgtgggtgagtttagccccatcccctaggtgttctccagcttgaggatcgcaggcagagaggaccagcccagcagccacaggcctgaccaaagcccaggctgggaaggagggcaactccccattttccactgggaggtgtttcacagcacagtcaacataggtgacctgcaaagatcctcatgtttgttattttctttggccagatccatccctacagggttcagcagggcctacaggaggggcagtgagagaacagaccccaaaaagaaaggggactccatgactgaccaccttgaggggggccaggctgcgggccccgttcatcttttttcattctcaggtcgctgatttcttggagcctgaaaagaaagtaacacagcagggatgaggacagatggtgtgagtcagtgagtgagtgacctgactaatagcctgggagggacagggcaggttttctgcagagcacggaagattcagctgaagtcagagaggtgaagccagtttcccagggtaacatagtgaggcactgaaagaaaggagactgcactggagcccaggtccccgggctccccagagctccttactcttcctcctcctcagcagcctggagaccccacaacctccagccggaggcctgaagcatgaggccatgccaggtgccaggtgatgctgggaattttcccgggagcttcgggtcttcccagcactctggtctcgcccgccctgcctctcgggctctgcccagcttcctgagtcctgacagagcacagtgggggagatgttggcagaggtggcagatgggctcacggccatccctcctgcaggagcagcgactggacccagagccatgtggctgtgccctctggccctcaacctcatcttgatggcagcctctggtgctgtgtgcgaagtgaaggacgtttgtgttggaagccctggtatccccggcactcctggatcccacggcctgccaggcagggacgggagagatggtctcaaaggagaccctggccctccaggtactgtgctgcagaccccaccctcagctgagggacacagaccccttttcaggaggcccatctgtccaggcccctaggctgtgggccatagtgagctgggggctatagtaagctgggtgggacttcagtctgcagggctggtgggttcctggggcccttatgatggcgcatcctggagagtctgtcctcatagtgcccacggagtgatagagtgatagctgagccagccctggtgataatgggcatcgagtctcactagctccaaccagttgtgggtgacagatcctacacatccatgtctcttttctctgcaggccccatgggtccacctggagaaatgccatgtcctcctggaaatgatgggctgcctggagcccctggtatccctggagagtgtggagagaagggggagcctggcgagaggggccctccaggtgagcagggtggggcaggtgggcagtggaaacatgggcacagcgaccctgaagtcagttacacggggatgatggggatcagacaaaccctacaggttccccaagggcatttggctcaacctaagtaagagaggataagcttgagggagaaagctgaggtgtctggggagtgtggtcacaattcagggaaaggcaggtgtgggaagtcctccgtgcctcatgaccaccgatggggacacactgagtcaggtgtgggatgagggacagcactgggaggcaggggaggcatgtcctgggatggaggccctgggggctgtctgaagggtgaatgcggacgaggcatccagacagacggtgtgatcaggagccccacagacagaggggaactttgaagctcagagcggtaagcaagtccatcagggcagtgcagagagcatcatgcttgcccttggtggagggtgcgggagagggacttgccccacagaggcgggcagacagaacccctcgagggacagagcaggaaagaggacaaggggtgggggtctcagcaggggcaaggcttcactaaagaataggggaccacggggtgtggagacacactggaatcttgtggaccctctgagcctagggtctgggtggcgcctaacagcaatgaaagggcagagttccaggattgcagatggcaaaacacctgcgtggcagcaagtgggagtcttcactggcctgcccctccttctgtgtggggcactctccacagggcttccagctcatctagatgaggagctccaagccacactccacgactttagacatcaaatcctgcagacaaggggaggtaaggggaccccctgggcctcacggggtaggagtttcccacaaattcccctcattctcagcaccagcttctagaacatagagattacaaataggcatgcacatgcaggtcttggggaaaggaatgacgcttgcttttctgatgtctttgaatggcccagaggagacagaagcagacacaattcactccccatttcataggaaagcaagttctccacctgccttgctttccactgaattccaggaaattgcaccatttctggcaataagtaattgttacttaggtgaatgaataaatggaggagagtctaaaagtgaatttagaaaactgcaattggaagaggaagagaagacacagagagaggcagagatggagagactggggagaatctggtagcagagaccccaggtgagggaggtggcttagagacaaagtggtcagtggcctgacccggactcctctgctctcagccctcagtctgcagggctccataatgacagtaggagagaaggtcttctccagcaatgggcagtccatcacttttgatgccattcaggaggcatgtgccagagcaggcggccgcattgctgtcccaaggaatccagaggaaaatgaggccattgcaagcttcgtgaagaagtacaacacatatgcctatgtaggcctgactgagggtcccagccctggagacttccgctactcagacgggacccctgtaaactacaccaactggtaccgaggggagcccgcaggtcggggaaaagagcagtgtgtggagatgtacacagatgggcagtggaatgacaggaactgcctgtactcccgactgaccatctgtgagttctgagaggcatttaggccatgggacagggaggacgctctctggccttcggcctccatcctgaggctccacttggtctgtgagatgctagaactccctttcaacagaattcacttgtggctattgggactggaggcacccttagccacttcattcctctgatgggccctgactcttccccataatcactgaccagccttgacactccccttgcaaactctcccagcactgcaccccaggcagccactcttagccttggccttcgacatgagatggagccctccttattccccatctggtccagttccttcacttacagatggcagcagtgaggtcttggggtagaaggaccctccaaagtcacacaaagtgcctgcctcctggtcccctcagctctctctctgcaacccagtgccatcaggatgagcaatcctggccaagcataatgacagagagaggcagacttcggggaagccctgactgtgcagagctaaggacacagtggagattctctggcactctgaggtctctgtggcaggcctggtcaggctctccatgaggttagaaggccaggtagtgttccagcagggtggtggccaagccaaccccatgattgatgtgtacgattcactcctttgagtctttgaatggcaactcagccccctgacctgaagacagccagcctaggcctctagggtgacctagagccgccttcagatgtgacccgagtaactttcaactgatgaacaaatctgcaccctacttcagatttcagtgggcattcacaccaccccccacaccactggctctgctttctcctttcattaatccattcacccagatatttcattaaaattatcacgtgccaggtcttaggatatgtcgtggggtgggcaaggtaatcagtgacagttgaagatttttttttcccagagcttatgtcttcatctgtgaaatgggaataagatacttgttgctgtcacagttattaccatccccccagctaccaaaattactaccagaactgttactatacacagaggctattgactgagcacctatcatttgccaagaaccttgacaagcacttctaatacagcatattatgtactattcaatctttacacaatgtcacgggaccagtattgtttcctcattttttataaggacactgaagcttggaggagttaaatgttttgagtattattccagagagcaagtggcagaggctggatccaaacccatcttcctggacctgaagcttatgcttccagccaccccactcctgagctgaataaagatgatttaagcttaataaatcgtgaatgtgttcaca

SEQ ID NO: 3 - DNA, 2230 bp, Homo sapiens
gacttggaggcagagacccaagcagctggaggctctgtgtgtgggtcgctgatttcttggagcctgaaaagaaagtaacacagcagggatgaggacagatggtgtgagtcagtgagagcagcgactggacccagagccatgtggctgtgccctctggccctcaacctcatcttgatggcagcctctggtgctgtgtgcgaagtgaaggacgtttgtgttggaagccctggtatccccggcactcctggatcccacggcctgccaggcagggacgggagagatggtctcaaaggagaccctggccctccaggccccatgggtccacctggagaaatgccatgtcctcctggaaatgatgggctgcctggagcccctggtatccctggagagtgtggagagaagggggagcctggcgagaggggccctccagggcttccagctcatctagatgaggagctccaagccacactccacgactttagacatcaaatcctgcagacaaggggagccctcagtctgcagggctccataatgacagtaggagagaaggtcttctccagcaatgggcagtccatcacttttgatgccattcaggaggcatgtgccagagcaggcggccgcattgctgtcccaaggaatccagaggaaaatgaggccattgcaagcttcgtgaagaagtacaacacatatgcctatgtaggcctgactgagggtcccagccctggagacttccgctactcagacgggacccctgtaaactacaccaactggtaccgaggggagcccgcaggtcggggaaaagagcagtgtgtggagatgtacacagatgggcagtggaatgacaggaactgcctgtactcccgactgaccatctgtgagttctgagaggcatttaggccatgggacagggaggacgctctctggccttcggcctccatcctgaggctccacttggtctgtgagatgctagaactccctttcaacagaattcacttgtggctattgggactggaggcacccttagccacttcattcctctgatgggccctgactcttccccataatcactgaccagccttgacactccccttgcaaactctcccagcactgcaccccaggcagccactcttagccttggccttcgacatgagatggagccctccttattccccatctggtccagttccttcacttacagatggcagcagtgaggtcttggggtagaaggaccctccaaagtcacacaaagtgcctgcctcctggtcccctcagctctctctctgcaacccagtgccatcaggatgagcaatcctggccaagcataatgacagagagaggcagacttcggggaagccctgactgtgcagagctaaggacacagtggagattctctggcactctgaggtctctgtggcaggcctggtcaggctctccatgaggttagaaggccaggtagtgttccagcagggtggtggccaagccaaccccatgattgatgtgtacgattcactcctttgagtctttgaatggcaactcagccccctgacctgaagacagccagcctaggcctctagggtgacctagagccgccttcagatgtgacccgagtaactttcaactgatgaacaaatctgcaccctacttcagatttcagtgggcattcacaccaccccccacaccactggctctgctttctcctttcattaatccattcacccagatatttcattaaaattatcacgtgccaggtcttaggatatgtcgtggggtgggcaaggtaatcagtgacagttgaagatttttttttcccagagcttatgtcttcatctgtgaaatgggaataagatacttgttgctgtcacagttattaccatccccccagctaccaaaattactaccagaactgttactatacacagaggctattgactgagcacctatcatttgccaagaaccttgacaagcacttctaatacagcatattatgtactattcaatctttacacaatgtcacgggaccagtattgtttcctcattttttataaggacactgaagcttggaggagttaaatgttttgagtattattccagagagcaagtggcagaggctggatccaaacccatcttcctggacctgaagcttatgcttccagccaccccactcctgagctgaataaagatgatttaagcttaataaatcgtgaatgtgttcacaaaaaaaaaaaaaaaa